(19) United States
(12) Patent Application Publication
(10) Pub. No.: US 2026/0137408 A1
(43) Pub. Date: May 21, 2026

(54) AXIALLY-ACTUATED MICRO-FORCEPS WITH FORCE-MODULATED JAWS

(71) Applicant: EyeMacular Regeneration, Inc., St. Paul, MN (US)

(72) Inventors: Timothy W. Olsen, Rochester, MN (US); John Christian Love, San Diego, CA (US)

(21) Appl. No.: 18/951,165

(22) Filed: Nov. 18, 2024

(51) Int. Cl. A61B 17/29

(52) U.S. Cl. CPC A61B 17/2909; A61B 2017/2901; A61B 2017/2944

(57) ABSTRACT

A medical device includes a sheath having a lumen and wall that extends along a length between ends, with an opening in its side near the tip. A movable shaft fits inside the sheath's lumen. The device has two jaws—one formed from a hanging portion of the sheath wall next to the side opening, and another formed at the shaft's tip. The jaws can move between closed and open positions as the shaft slides within the sheath. A mechanism controls the squeezing force between the jaws. Both jaws are sized to fit through very small openings, with each jaw measuring less than 0.51 millimeters across when open.

### TECHNICAL FIELD

This document relates to micro-scale medical forceps having axially-actuated jaws with force modulation for grasping an object through a small-diameter cannula.

### BACKGROUND

Macular degeneration is a leading cause of vision loss for people over age 50. Effective treatment remains elusive. One approach includes a transplantation procedure. Complexities associated with transplantation include difficulty in manipulating the excised tissue. A carrier structure can provide support for the tissue however, manipulating the carrier structure, in the region of the surgical site, can be challenging. A traditional jointed forceps is generally unworkable because it requires clearance for manipulating the jaws and requires an unsuitably large access port.

### SUMMARY

An example of the present subject matter includes a micro-scale medical clamping device. In one example, a device includes a sheath having a lumen and wall extending along a longitudinal axis. The sheath has a lateral opening near a distal end. A shaft is disposed within the lumen and is configured for bidirectional axial movement. A first jaw is formed from a pendent segment of the wall adjacent to the lateral opening and a second jaw is provided at the shaft's distal end. The second jaw includes a protruding element that retracts into the lumen in a separated position and ejects through the lateral opening in an approximated position. With the jaws in a separated position, the cross-sectional dimension of both jaws is sufficiently small to allow the device to pass through an orifice having a diameter compliant with vitreoretinal and intraocular surgical access. In one example, the maximum cross section dimension, measured over the jaws while in a separated position, is less than 0.51 mm.

Conventional forceps designs utilize pivoting handles and jaws connected by a joint, similar to scissors. However, this traditional configuration prevents the device from achieving the compact dimensions needed for passage through small-gauge cannulas used in vitreoretinal microsurgery. The mechanical constraints of these pivoting jaws necessitate an access port that exceeds acceptable size limitations for delicate ophthalmic procedures.

Moreover, conventional forceps designs suffer from a direct relationship between handle force and jaw clamping pressure. Without force modulation, the compression force applied at the jaws directly corresponds to the manual pressure exerted on the handles. This uncontrolled force transmission creates a significant risk of damaging delicate tissues or carrier structures being manipulated during microsurgical procedures.

In contrast to a typical forceps, an example of the disclosed subject matter includes a shaft and sheath arranged substantially coaxially and configured for relative motion aligned in the axial direction. In this manner, an example of the present subject matter can be viewed as vertically acting forceps.

A force modulation mechanism of the device controls a compressive force exerted between the jaws. The force modulation mechanism can include a resilient component which limits the compressive force that can be applied by the jaws. An actuator affixed to the proximal end of a device includes both a handle and an operable component that can be coupled to the sheath and shaft and induce relative axial movement when manipulated by an operator.

Various force modulation mechanisms can be provided. For example, an actuator can include a helical spring that deflects with continued manipulation of the handle and dampens a compressive force at the jaws. In addition to a helical spring, other resilient mechanisms can include a shape-memory material, a leaf spring, an elastomeric element, or other compressible components.

A manufacturing method can include forming a lateral opening in a sheath and configuring a pendent segment in the manner of a first jaw. The method can include providing a force modulation mechanism in a system associated with an actuator, a shaft, a sheath, or the jaws. The manufacturing method can include configuring the jaws for relative movement and for deployment and retraction to allow passage through a small dimension.

Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 includes an illustration of a device and actuator, according to one example.

FIG. 2A-2C include illustrations of a portion of a device with an object, according to one example.

FIG. 2D illustrates a section view of a portion of a device with an object, according to one example.

FIGS. 3A-3E illustrate section views of a portion of a device with an object, according to one example.

FIG. 4 illustrates a section view of an actuator, according to one example.

FIGS. 5A-5C illustrate views of a device, according to various examples.

FIG. 6 illustrates a flowchart of a method, according to one example.

### DETAILED DESCRIPTION

FIG. 1 includes an illustration of system 100A including device 110A and actuator 50A, according to one example. Device 110A includes a linear segment having a proximal end coupled to actuator 50A at a proximal end 125A. In the figure, object 20 is grasped by jaws disposed at distal end 127A.

Operator manipulation of actuator 50A can include modulating a girth of a plurality of elongate members arranged about a core. By alternately squeezing or relaxing actuator 50, the jaws at distal end 127 alternately move between an open position and a closed position, here referred to as separated position and approximate position, respectively.

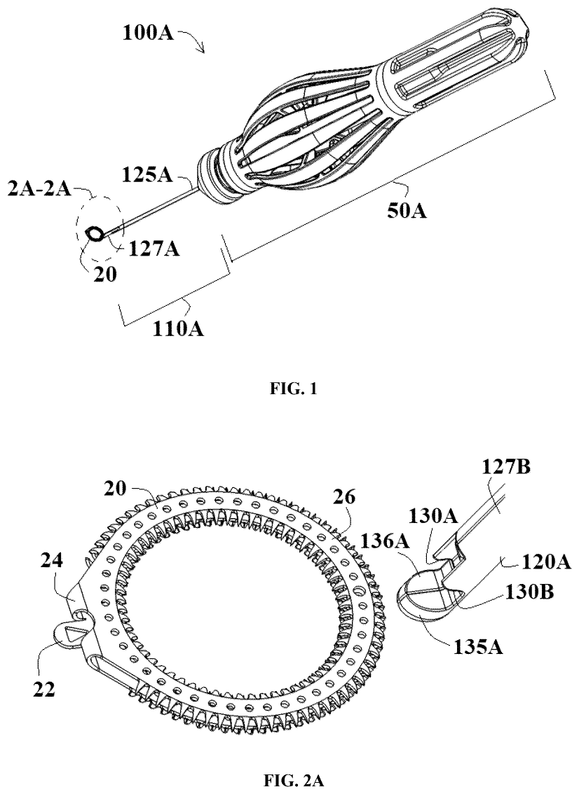

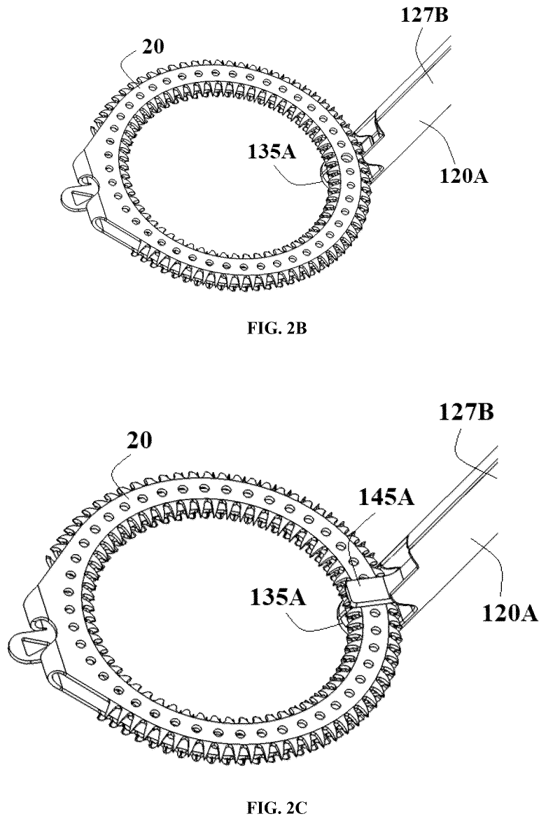

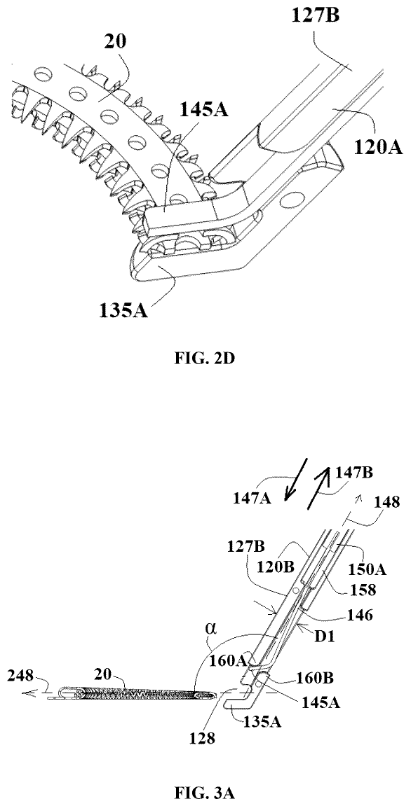

FIG. 2A illustrates an enlarged view of distal end 127B and object 20, according to one example. Object 20, in this example, includes a pair of rings coupled by joint 24. A surgical transplantation or translocation procedure includes positioning donor tissue between the rings and includes grasping and manipulating the rings using, in part, pendent segment 22. Under certain conditions, pendent segment 22 does not provide sufficient control and stability for transplantation or location of object 20. When object 20 carries donor tissue, an excess clamping force on the two ring segments may result in crush injury to the tissue. A fluidic gelatinous, or gas-filled intraocular environment within the surgical site can complicate precise control of the tissue carrier structure and restrict delicate surgical maneuvers.

Here is a suggested edit to improve clarity:

An example of a device as described herein facilitates tissue transplantation procedures by providing a stable gripping platform. This platform serves two functions: First, it enables precise manipulation of the carrier structure when transferring tissue between surgical instruments. Second, it acts as a secure anchoring point during tissue placement at the target site. The device's robust clamping mechanism creates a stable reference point against which other surgical instruments can be coordinated for precise tissue manipulation and placement.

An example of the present subject matter includes a device configured to suitably grasp object 20 at a position displaced from pendent segment 22. For example, a device according to the present subject matter can grasp object 20 at position 26 disposed opposite or complementary to the location of pendent segment 22, as shown in FIG. 2A.

As shown in FIG. 2A, distal end 127B includes sheath 120A. Sheath 120A, in this example, has walls configured to form shoulders 130A and 130B contoured to engage with a surface of object 20 and provide a measure of stability. In addition, sheath 120A is terminated with first jaw 135A having first jaw face 136A. As shown, first jaw 135A is formed by a lateral opening in a wall of sheath 120A. In this example, first jaw 135A is fixedly coupled to sheath 120A.

FIG. 2B illustrates a view of distal end 127B positioned for engagement with object 20. In this view, first jaw 135A is visible beneath a portion of object 20. Object 20 is positioned in a lateral opening of sheath 120A.

FIG. 2C illustrates a view of distal end 127B proximate object 20. In this view, object 20 is disposed between first jaw 135A (visible beneath a portion of object 20) and second jaw 145A. Second jaw 145A is shown above a portion of object 20. Second jaw 145A, as shown here, includes a rectangular segment having a planar surface that aligns with a surface of the object 20.

FIG. 2D illustrates a section view of a portion of device 120A with object 20, according to one example. In this view, distal end 127B includes sheath 120A coupled to first jaw 135A, which engages with a lower ring of object 20. In addition, distal end 127B includes a shaft terminating in second jaw 145A, which engages with an upper ring of object 20. Second jaw 145A includes portion of flat stock having a protruding element that emerges from the lateral opening in sheath 120A, and applies a vertically acting force on the carrier with force limitations.

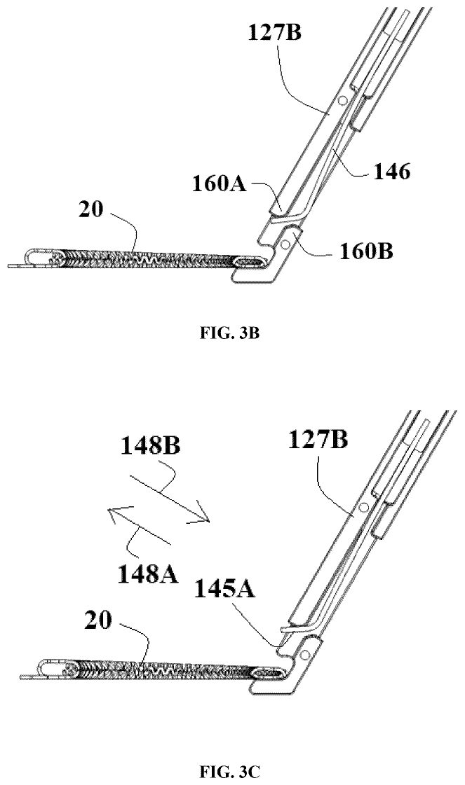

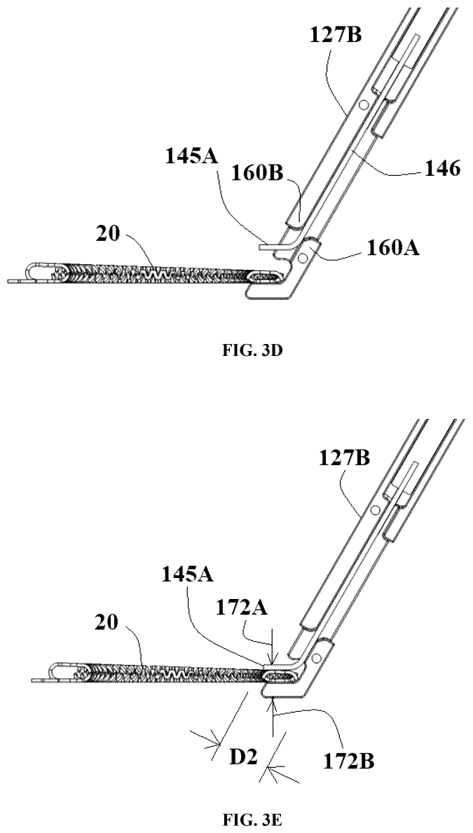

FIGS. 3A through 3E illustrates a sequence of side views that illustrate one example of the subject matter. In FIG. 3A, distal end 127B includes sheath 120B and shaft 150A. Shaft 150A includes a distal end that is coupled to second jaw 145A by second jaw coupler 146. Shaft 150A is disposed in lumen 158 within sheath 120B and is aligned on longitudinal axis 148.

The figure illustrates lateral opening 128 and first jaw 135A formed of a pendent segment sheath 120. Second jaw coupler 146 is shown to be deflected away from alignment with axis 148 by force of interference between second jaw 145A and guide element 160A formed on an interior surface of a portion of lumen 158. Object 20 lies in a plane denoted by axis 248. Angle α, between axis 248 and axis 148, provides good access as between the device and the object during a particular surgical procedure. For example, angle α can be substantially 120 degrees (substantial understood to be ±20% of the stated nominal value). Shaft 150A is configured for bidirectional relative motion as shown by arrow 147A and arrow 147B.

FIG. 3B, viewed in conjunction with the illustration in FIG. 3A, illustrates relative motion whereby shaft 150A has advanced in the direction of arrow 147A relative to a stationary sheath 120B. In this example, shown, provided the sheath (and first jaw 135A) remain stationary, and shaft 150A (and second jaw 145A) translates in the direction of arrow 147A, by virtue of guide 160A, second jaw 145A emerges from sheath 120B, and projects from lateral opening 128. FIG. 3C illustrates further progression whereby second jaw 145A has translated both in the axial direction, as denoted by arrow 147A, and translated in a radial direction, as denoted by arrow 148A. Arrow 148A represents a radial direction with respect to longitudinal axis 148. As shown in FIGS. 3B and 3C, object 20 abuts first jaw 135. FIG. 3D depicts further progression following FIG. 3C in which second jaw is deflected, and supported, to a greater radial position via guide 160B. In FIG. 3E, second jaw 145A contacts object 20 and with further advancement, exerts a compressive force represented here by arrows 172A and 172B.

Among other things, the sequence of images depicted in FIGS. 3A-3E illustrate the movement of second jaw 145B coincident with relative motion as between shaft 150A and sheath 120A. In addition, when in the separated position (as shown in FIG. 3A, for example), a major diameter measured transverse to the longitudinal axis, is represented by dimension D1.

In one example, dimension D1 is sized to allow passage through a lumen of diameter less than 0.51 mm, which coincides with a size 23-gauge vitrectomy cannulae (having a nominal inner diameter of 0.50 to 0.51 mm and nominal outside diameter of 0.6 to 0.64 mm). Other values for dimension D1 are also contemplated, including, for example, size 25-gauge vitrectomy cannulae has a nominal inner diameter of 0.39 to 0.40 mm (and a nominal outer diameter of 0.51 to 0.52 mm) and a size 27-gauge vitrectomy cannulae having a nominal inner diameter of 0.25 to 0.26 mm (and a nominal outer diameter of 0.41 to 0.42 mm).

Furthermore, when in the approximate position (as shown in FIG. 3E, for example), a major diameter measured transverse to the longitudinal axis, is represented by dimension D2. In one example, dimension D2, larger than that of dimension D1, is sized to provide good retention of object 20 between first jaw 135A and second jaw 145.

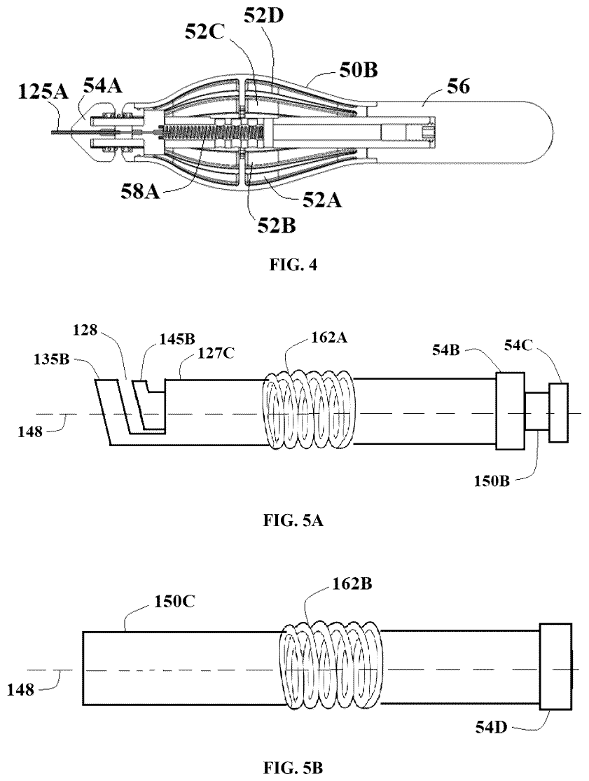

FIG. 4 illustrates a section view of actuator 50B, according to one example. Actuator 50B includes collet 54A and a plurality of linear elements, here denoted as a plurality of actuation members, some examples of which are denoted as actuation members 52A, 52B, 52C, and 52D. Actuation members 52A, 52B, 52C, and 52D are coupled to a handle member, or core 56. In the figure, proximal end 125A is coupled to collet 54A. In addition, resilient member 58A establishes a bias in which the actuation members, in a state of rest, are deflected as shown, and when displaced by a force, such as a manually exerted compression about the core, resilient member 58A (such as a helical wound spring), collapses and induces relative motion between the shaft and sheath coupled to collet 54A.

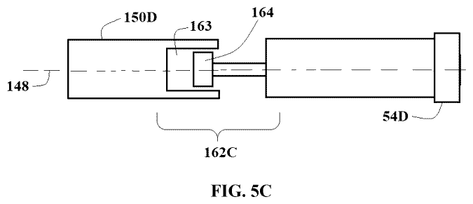

FIG. 5A illustrates a view of shaft 150B disposed in a lumen of sheath 127C aligned on axis 148. In this example, collet 54B is coupled to sheath 127C and collet 54C is coupled to shaft 150B. As illustrated, lateral opening 128 is disposed proximate distal end 127, thus forming first jaw 135B.

FIG. 5B illustrates shaft 150C having a resilient portion 162A disposed on a length of shaft 150C. In this example, second jaw 145B is depicted as a radiused shaft end.

FIG. 5C illustrates shaft 150D having resilient portion 162B disposed on a length of shaft 150D. In this example, resilient portion 162B includes piston 164 configured for movement in cylinder 163. In various examples a fluid within cylinder 163 is displaced by movement of piston 164 during which fluid is discharged through an orifice or in a gap between the piston 164 and the wall of cylinder 163, and provides dampened movement force exerted at second jaw 145C. In the example shown, second jaw 145C includes a conical surface aligned to harmonize with an angular orientation of first jaw.

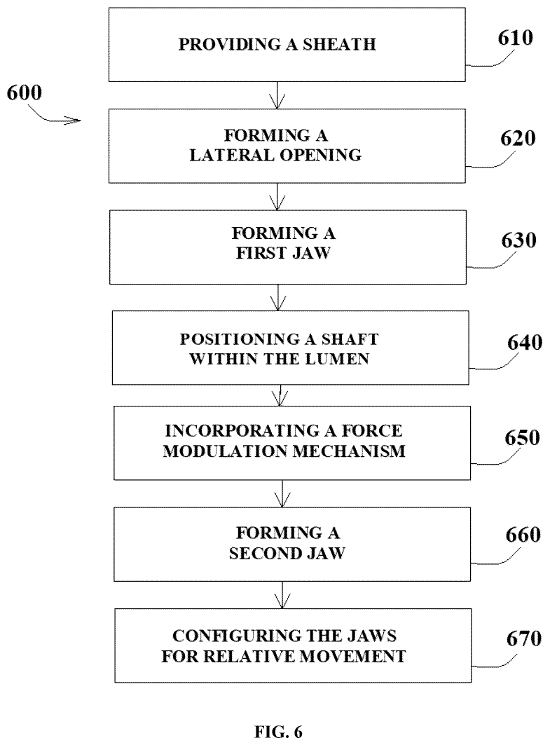

FIG. 6 illustrates a flowchart of method 600, according to one example. Method 600 represents an example of a method for manufacturing a clamping device.

At 610, method 600 includes providing a sheath. The sheath can include a tubular member having a wall and a lumen extending along a longitudinal axis between a proximal end and a distal end.

At 620, method 600 includes forming a lateral opening in the sheath near the distal end. The lateral opening can be formed in the sheath wall in a manner to provide clearance for receiving an object to be grasped.

At 630, method 600 includes forming a first jaw. The first jaw can be formed by configuring a pendent segment of the wall adjacent the lateral opening. The first jaw can have a jaw face configured to engage an object.

At 640, method 600 includes positioning a shaft within the lumen. The shaft is shaft configured for bidirectional axial movement relative to the sheath.

At 650, method 600 includes incorporating a force modulation mechanism configured to control a compressive force between the first jaw and a second jaw. The force modulation mechanism can be coupled to the first jaw, the second jaw, the shaft, the sheath, or the actuation mechanism.

At 660, method 600 includes forming a second jaw. The second jaw can include a distal end of the shaft and can include a protruding element that can retract to accommodate passage through a maximum dimensional constraint and can deploy to engage an object.

At 670, method 600 includes configuring the first jaw and the second jaw for relative movement. The jaws can be configured by for relative movement based on an axial movement of the shaft relative to the sheath.

In one example, the compressive force exerted by the device is limited to a value that avoids crushing or distorting the tissue or the tissue carrier structure. For example, a compression force is limited to less than 1 N.

The resilient member can be provided by a helical coil spring, an elastomeric material, or a fluid moving through an orifice or in an arrangement of a piston in a cylinder. The resilient member can be in a first jaw, a second jaw, a shaft, a sheath, or in an actuator. For example, in a shaft, the resilient member can include a shaped leaf spring or a torsion spring.

An example of the claimed subject matter can be fabricated of various materials. For example, a metal alloy, such as stainless steel, can be used. In addition, selected components can be fabricated by any of a number of polymer materials.

One example includes a miniature clamping device and system particularly suited for microsurgical applications. A device includes a sheath with a lumen extending along a longitudinal axis between proximal and distal ends. Near the distal end, the sheath includes a lateral opening in its wall. A shaft is disposed within the lumen and can move bidirectionally along the axis relative to the sheath.

A lateral opening can include a gap or an aperture in the wall of the sheath positioned near its distal end. This opening serves a functional purpose by allowing the pendent segment (which forms the first jaw) to be created from the sheath wall material adjacent to the opening. The lateral orientation indicates that the opening is positioned on the side of the sheath rather than at its end, and its placement is proximate to (near) the distal end of the sheath.

The first jaw can be viewed as a pendent segment of the sheath wall that is formed adjacent to the lateral opening. The term ‘pendent’ indicates that this jaw portion hangs or extends from the main sheath wall structure. In one example, the jaw is not a separate component attached to the sheath, but rather is integrally formed from the sheath wall material itself. Furthermore, when measured perpendicular to the device's longitudinal axis, the pendent jaw segment has a maximum cross-sectional dimension of less than 0.51 mm when in its separated position. The jaw is capable of moving between approximated and separated positions through its interaction with the shaft's axial movement.

The device includes a jaw arrangement where a first jaw is formed from a pendent segment of the sheath wall adjacent to the lateral opening. A second jaw is formed at the distal end of the internal shaft and includes a protruding element. The jaws can move between an approximated (closed) position and a separated (open) position based on the axial position of the shaft relative to the sheath. When separated, the protruding element of the second jaw retracts into the lumen. When approximated, it ejects through the lateral opening to engage with the first jaw.

A force modulation mechanism controls the compressive force between the jaws. This mechanism can include resilient segments made from materials such as shape-memory alloys, springs, elastomeric elements, or other compressible components. The first jaw includes a jaw face that can be planar and oriented at a specific angle (e.g., 120 degrees) relative to the longitudinal axis. The second jaw's protruding element can, for example, be configured as a rectangular flat tang projecting from a round shaft, oriented parallel to the first jaw's face.

In one example, the device is very small size-both jaws and the sheath have maximum cross-sectional dimensions of less than 0.51 mm when measured perpendicular to the longitudinal axis. The jaws can exert variable clamping force, and the lumen includes guide elements to direct the second jaw's movement.

The system includes an actuator coupled to the proximal ends of both sheath and shaft, including an actuation member connected to a handle portion. The relative movement between these components controls the axial displacement of the sheath relative to the shaft, thereby operating the jaws.

A manufacturing method involves providing the sheath, forming the lateral opening and pendent first jaw, positioning the shaft within the lumen, incorporating the force modulation mechanism, and forming the second jaw with its protruding element. The process ensures all components maintain the specified sub-0.51 mm cross-sectional dimension requirement.

### Various Notes

1The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. 1Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed:

1. A device comprising:
a sheath having a lumen and a wall extending along a longitudinal axis between a proximal end and a distal end, the sheath including a lateral opening proximate the distal end;
a shaft disposed within the lumen and configured for bidirectional axial movement relative to the sheath, the shaft having a shaft proximal end and a shaft distal end;
a first jaw comprising a pendent segment of the wall adjacent the lateral opening;
a second jaw comprising the shaft distal end and including a protruding element;
a force modulation mechanism configured to control a compressive force between the first jaw and the second jaw;
wherein the first jaw and the second jaw are relatively movable between an approximated position and a separated position based on an axial position of the shaft relative to the sheath, wherein the protruding element is configured to retract into the lumen when in the separated position and eject from the lateral opening when in the approximated position; and
wherein each of the first jaw and the second jaw has a maximum cross-sectional dimension, measured perpendicular to the longitudinal axis when in the separated position, of less than 0.51 mm.

2. The device of claim 1, wherein the force modulation mechanism includes a resilient segment of at least one of the shaft, the sheath, the first jaw, and the second jaw.

3. The device of claim 2, wherein the resilient segment includes at least one of a shape-memory material, a helical spring, a leaf spring, an orifice, a fluid, an elastomeric element, a compressible component, and a magnet.

4. The device of claim 1, wherein the first jaw includes a first jaw face and the second jaw includes a second jaw face.

5. The device of claim 4, wherein the first jaw face is planar and oriented at an angular orientation relative to the longitudinal axis.

6. The device of claim 4, wherein the first jaw face is oriented at an angle of 120 degrees relative to the longitudinal axis.

7. The device of claim 4, wherein the second jaw face comprises a cylindrical engagement surface having a uniform diameter.

8. The device of claim 4, wherein the protruding element includes a rectangular flat tang projecting from a round shaft.

9. The device of claim 8, wherein the protruding element is oriented parallel to a plane of the first jaw face.

10. The device of claim 1, wherein the sheath has an outside diameter of less than 0.51 mm.

11. The device of claim 1, wherein the first jaw and the second jaw can exert a variable clamping force therebetween.

12. The device of claim 1, wherein the lumen includes a guide element configured to guide the second jaw along a path in a radial direction relative to the longitudinal axis during axial movement of the shaft.

13. A clamping system comprising:
a sheath having a lumen and a wall extending along a longitudinal axis between a proximal end and a distal end, the sheath including a lateral opening proximate the distal end;
a shaft disposed within the lumen and configured for bidirectional axial movement relative to the sheath, the shaft having a shaft proximal end and a shaft distal end forming a second jaw, the second jaw including a protruding element;
a first jaw comprising a pendent segment of the wall adjacent the lateral opening;
a force modulation mechanism configured to control a compressive force between the first jaw and the second jaw;
wherein the first jaw and the second jaw are configured for relative movement between an approximated position and a separated position based on relative movement of the sheath and the shaft, wherein the protruding element is configured to retract into the lumen when in the separated position and eject from the lateral opening when in the approximated position;
wherein each of the first jaw and the second jaw has a maximum cross-sectional dimension, measured perpendicular to the longitudinal axis when in the separated position, of less than 0.51 mm; and
an actuator coupled to the proximal end of the sheath and to the shaft, the actuator comprising an actuation member connected to a handle portion, wherein relative movement between the actuation member and the handle portion is configured to axially displace the sheath relative to the shaft.

14. The clamping system of claim 13, wherein the force modulation mechanism includes a resilient segment coupled to at least one of the shaft, the sheath, the first jaw, the second jaw, and the actuator, wherein the resilient segment comprises at least one of a shape-memory material, a helical spring, a leaf spring, an orifice, a fluid, an elastomeric element, a compressible component, and a magnet.

15. The clamping system of claim 13, wherein the sheath has an outside diameter of less than 0.51 mm.

16. The clamping system of claim 13, wherein the actuator is coupled to the proximal end of the sheath and to the shaft via a securing mechanism.

17. A method of manufacturing a clamping device, the method comprising:
providing a sheath having a lumen and a wall extending along a longitudinal axis between a proximal end and a distal end;
forming a lateral opening in the sheath proximate the distal end;
forming a first jaw comprising a pendent segment of the wall adjacent the lateral opening;
positioning a shaft within the lumen, the shaft configured for bidirectional axial movement relative to the sheath;
incorporating a force modulation mechanism configured to control a compressive force between the first jaw and a second jaw;
forming the second jaw comprising a distal end of the shaft and including a protruding element;
configuring the first jaw and the second jaw for relative movement between an approximated position and a separated position, wherein the protruding element is configured to retract into the lumen when in the separated position and eject from the lateral opening when in the approximated position; and
wherein each of the first jaw and the second jaw has a maximum cross-sectional dimension, measured perpendicular to the longitudinal axis when in the separated position, of less than 0.51 mm.

18. The method of claim 17, wherein forming the first jaw includes creating a jaw face with a planar surface at an angular orientation relative to the longitudinal axis.

19. The method of claim 17, wherein forming the second jaw includes forming a round stud aligned with the first jaw, the round stud having a flat end, a semispherical ball-end, or a conical end.

20. The method of claim 17, wherein incorporating the force modulation mechanism includes providing a resilient segment on the shaft.
